(19) United States
(12) Patent Application Publication
(10) Pub. No.: US 2008/0233135 A1
(43) Pub. Date: Sep. 25, 2008

(54) Cobalamin taxane bioconjugates

(76) Inventors: John R. Gebhard, Salt Lake City, UT (US); David Vollmer, West Jordan, UT (US); Claire Daugherty, Salt Lake City, UT (US); Dinesh Patel, Salt Lake City, UT (US)

Correspondence Address: THORPE NORTH & WESTERN, LLP., P.O. Box 1219, SANDY, UT, 84091-1219, US

(21) Appl. No.: 12/077,060

(22) Filed: Mar. 14, 2008

Related U.S. Application Data
(60) Provisional application No. 60/919,121, filed on Mar. 19, 2007.

(51) Int. Cl. A61K 47/48; A61K 31/714; C07H 23/00; A61P 35/00

(52) U.S. Cl. 424/178.1; 514/52; 536/26.4; 530/387.1

(57) ABSTRACT

The present invention is directed to methods and compositions including a taxane covalently bonded to the cobalt atom of a cobalamin. The composition can be delivered by any effective route, but is particularly useful as an oral anti-cancer or antiangiogenic compound. The anti-cancer/anti-angiogenic compound can be used in various chemotherapies including anti-angiogenic chemotherapies, alone or in combination with other anti-cancer/anti-angiogenic compounds.

The present non-provisional application claims the benefit of U.S. Provisional Application No. 60/919,121, filed Mar. 19, 2007, which is incorporated herein by reference.

### BACKGROUND

The efficacy of certain drugs in treating disease is often dependent on how readily an effective amount of the drug can be delivered to a specific location in a subject's body, particularly to a specific type of tissue or population of cells. Insuring that a drug or active agent is mainly utilized by the appropriate cells may impart a number of benefits, e.g. achieving efficacy with smaller doses, decreasing non-targeted cytotoxicity, and decreased impact on a subject's renal system. Therefore, methods and compositions that facilitate drug targeting can be of considerable value to the pharmaceutical and medicinal arts. One approach to this need involves using molecules that have generally understood transport mechanisms and which can be induced to release drugs in site-specific fashion.

One such mechanism involves the use of cobalamin (Cbl). Cobalamin is an essential biomolecule, the size of which prevents it from being taken up from the intestine and into cells by simple diffusion, but rather by facultative transport. Cobalamin must bind to a specific protein, and the complex may is actively taken up through a receptor-mediated transport mechanism. In the small intestine, cobalamin binds to intrinsic factor (IF) secreted by the gastric lining. The Cbl-IF complex binds to IF receptors on the lumenal surface of cells in the ileum and is transcytosed across these cells into the bloodstream. Once there, cobalamin binds to one of three transcobalamins (TCs) to facilitate its uptake by cells. The receptor-mediated nature of cobalamin uptake imparts a degree of cell-specificity to cobalamin metabolism, in that cobalamin will only be absorbed and metabolized by cells that present the correct receptor(s). This specificity has been exploited in targeting drugs or other active agents to certain cell types. By conjugating an agent to cobalamin directly or indirectly, one can facilitate its preferred absorption by cells that utilize cobalamin heavily.

Several patents have utilized cobalamin for various purposes. For example, Grissom et al. has obtained several patents: U.S. Pat. Nos. 6,790,827; 6,777,237; and 6,776,976; using organocobalt complexes. Russell-Jones et al. has also utilized cobalamin to increase uptake of active agents, as described in a series of patents, including 5,863,900; 6,159,502; and 5,449,720. In addition to this, research and development for methods and compositions having increased bioavailability of various pharmaceutical agents continue to be sought.

### SUMMARY

It has been recognized that it would be advantageous to develop compositions and methods for delivery of taxanes. Briefly, and in general terms, the invention is directed to methods and compositions including a taxane covalently bonded to the cobalt atom of a cobalamin as a cobalamin-taxane bioconjugate. In one embodiment, paclitaxel is covalently bonded to the cobalt atom of a hydroxycobalamin, or more generally, one of the various forms of vitamin B₁₂. In another embodiment, the bioconjugate can be formulated as a composition with another anti-cancer compound. In yet another embodiment, a cobalamin-taxane bioconjugate can have a water solubility of at least 0.5 mg/ml, or even over 100 mg/ml. Methods of administering and/or treating cancer include administering a cobalamin-taxane conjugate as an oral, parenteral, or dermal composition in a chemotherapy or anti-angiogenic regimes, either using maximum tolerated dosing or metronomic dosing, for example.

Additional features and advantages of the invention will be apparent from the detailed description which follows, taken in conjunction with the accompanying drawings, which together illustrate, by way of example, features of the invention.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

In describing and claiming the present invention, the following terminology will be used in accordance with the definitions set forth below.

It must be noted that, as used in this specification and the appended claims, the singular forms “a,” “an,” and, “the” include plural referents unless the context clearly dictates otherwise. Thus, for example, reference to “a taxane” can include one or more of such taxanes, reference to “an amount of anti-cancer compounds” can include reference to one or more amounts of anti-cancer compounds, and reference to “the cobalamin” can include reference to one or more cobalamins.

As used herein, the terms “formulation” and “composition” can be used interchangeably and refer to at least one pharmaceutically active agent, such as a taxane covalently bonded to the cobalt atom of a cobalamin with a covalent linkage. The terms “drug,” “active agent,” “bioactive agent,” “pharmaceutically active agent,” and “pharmaceutical,” can also be used interchangeably to refer to an agent or compound that has measurable specified or selected physiological activity when administered to a subject in an effective amount. As used herein, “carrier” or “inert carrier” refers to typical compounds or compositions used to carry drugs, such as polymeric carriers, liquid carriers, or other carrier vehicles with which a bioactive agent may be combined to achieve a specific dosage form. As a general principle, carriers do not substantially react with the bioactive agent in a manner that substantially degrades or otherwise adversely affects the bioactive agent or its therapeutic potential.

As used herein, “administration,” and “administering” refer to the manner in which a drug, formulation, or composition is introduced into the body of a subject. Various art-known routes such as oral, parenteral, transdermal, and transmucosal can accomplish administration. Thus, an oral administration can be achieved by swallowing, chewing, dissolution via adsorption to a solid medium that can be delivered orally, or sucking an oral dosage form comprising active agent(s). Parenteral administration can be achieved by injecting a drug composition intravenously, intra-arterially, intramuscularly, intrathecally, or subcutaneously, etc. Transdermal administration can be accomplished by applying, pasting, rolling, attaching, pouring, pressing, rubbing, etc., of a transdermal preparation onto a skin surface. Transmucosal administration may be accomplished by bringing the composition into contact with any accessible mucous membrane for an amount of time sufficient to allow absorption of a therapeutically effective amount of the composition. Examples of transmucosal administration include inserting a suppository into the rectum or vagina; placing a composition on the oral mucosa, such as inside the cheek, on the tongue, or under the tongue; or inhaling a vapor, mist, or aerosol into the nasal passage. These and additional methods of administration are well known in the art.

The term “effective amount,” refers to an amount of an ingredient which, when included in a composition, is sufficient to achieve an intended compositional or physiological effect. Thus, a “therapeutically effective amount” refers to a non-lethal amount of an active agent sufficient to achieve therapeutic results in treating a condition for which the active agent is known or taught herein to be effective. Various biological factors may affect the ability of a substance to perform its intended task. Therefore, an “effective amount” or a “therapeutically effective amount” may be dependent on such biological factors. Further, while the achievement of therapeutic effects may be measured by a physician or other qualified medical personnel using evaluations known in the art, it is recognized that individual variation and response to treatments may make the achievement of therapeutic effects a subjective decision. In some instances, a “therapeutically effective amount” of a drug can achieve a therapeutic effect that is measurable by the subject receiving the drug. For example, in metronomic dosing, “the “therapeutic effective amount” may increase or decrease during the therapeutic treatment due to inherent genetic variation. The determination of an effective amount is well within the ordinary skill in the art of pharmaceutical, medicinal, and health sciences.

As used herein, “treat,” “treatment,” or “treating” refers to the process or result of giving medical aid to a subject, where the medical aid can counteract a malady, a symptom thereof, or other related adverse physiological manifestation. Additionally, these terms can refer to the administration or application of remedies to a patient or for a disease or injury; such as a medicine or a therapy. Accordingly, the substance or remedy so applied, such as the process of providing procedures or applications, are intended to relieve illness or injury. As used herein, “reduce” or “reducing” refers to the process of decreasing, diminishing, or lessening, as in extent, amount, or degree of that which is reduced. Additionally, the use of the term can include from any minimal decrease to absolute abolishment of a physiological process or effect.

As used herein, “subject” refers to an animal, such as a mammal, that may benefit from the administration of an anti-cancer agent and/or bioconjugate compound, including formulations or compositions that include such an agent and/or compound.

As used herein, the term “taxane” generally refers to a class of diterpenes produced by the plants of the genus Taxus (yews). This term also includes those taxanes that have been artificially synthesized. For example, this term includes paclitaxel and docetaxel, and derivatives thereof.

As used herein, the term “cobalamin” refers to an organocobalt complex having the essential structure shown below:

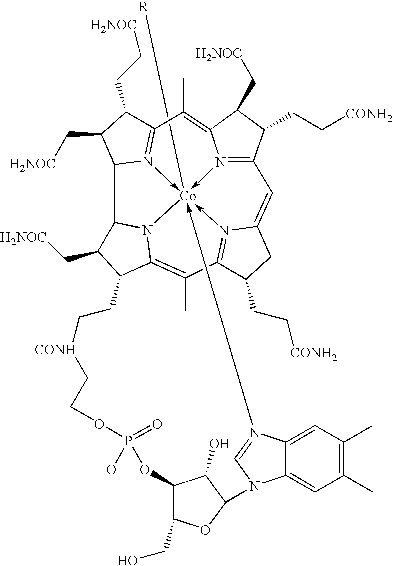

as well as derivatives of this structure in which R may be —CH₃ (methylcobalamin), —CN (cyanocobalamin), —OH (hydroxycobalamin), —C₁₀H₁₂N₅O₃ (deoxyadenosylcobalamin), or synthetic complexes that include a corrin ring and are recognized by cobalamin transport proteins, receptors, and enzymes. The term also encompasses inclusion of substituent groups on the corrin ring that do not eliminate its binding to transport proteins. The term “organocobalt complex” refers to an organic complex containing a cobalt atom having bound thereto 4-5 calcogens as part of a multiple unsaturated heterocyclic ring system, particularly any such complex that includes a corrin ring.

The organocobalt molecule cobalamin is an essential biomolecule with a stable metal-carbon bond. Among other things, cobalamin plays a role in the folate-dependent synthesis of thymidine, an essential building block of DNA. Because cobalamin is a large molecule, cellular uptake of cobalamin is achieved by receptor-mediated endocytosis. The density of receptors in a cell may be modulated in accordance with the cell's need for cobalamin at a given time. For example, a cell may upregulate its expression of cobalamin receptors during periods of high demand for cobalamin. One such time is when the cell replicates its DNA in preparation for mitosis or meiosis. One result of this facultative upregulation is that cobalamin uptake will be higher in cell populations undergoing rapid proliferation than in slower-growing cell populations. This non-uniform uptake profile makes it possible to target delivery of a bioactive agent to high-demand cell populations by linking the agent to cobalamin.

Cobalamin is the most chemically complex of the vitamins. The core structure of the cobalamin molecule is a corrin ring consisting of four pyrrole subunits, two of which are directly connected with the remainder connected through a methylene group. Each pyrrole has a proprionamide substituent that extends radially from the ring. At the center of the ring is a cobalt atom in an octahedral environment that is coordinated to the four corrin ring nitrogens, as well as the nitrogen of a dimethylbenzimidazole group. The sixth coordination partner can vary as previously discussed; represented by R in formula I. Six propionamide groups extend from the outer edge of the ring, while a seventh links the dimethylbenzimidazole group to the ring through a phosphate group and a ribose group.

The term “vitamin B₁₂” or “B₁₂” has been generally used in two different ways in the art. In a broad sense, it has been used interchangeably with four common cobalamins: cyanocobalamin, hydroxocobalamin, methylcobalamin, and adenosylcobalamin. In a more specific way, this term refers to only one of these forms, cyanocobalamin, which is the principal B₁₂ form used for foods and in nutritional supplements. For the purposes of this invention, this term includes cyanocobalamin, hydroxocobalamin, methylcobalamin, and adenosylcobalamin, unless the context dictates otherwise.

As used herein the term “bioconjugate” refers to a molecule containing a taxane covalently bonded directly to the cobalt of cobalamin or is bound indirectly to the cobalt of cobalamin via a covalent linkage. As the bioconjugates provided herein have been shown to have anti-angiogenic properties and as cobalamin bioconjugates have been shown to have anti-cancer properties in the art, the term “bioconjugate” has been used to refer to “anti-cancer” and “anti-angiogenic” compounds herein.

As used herein “anti-cancer compound” refers to any compound, drug, agent, or molecule that can be used in cancer treatments. This term includes the cobalamin-taxane bioconjugates disclosed by the present invention as well as other known anti-cancer agents and drugs, including those found in Gordon M. Cragg, David G. I. Kingston, & David J. Newman, Anticancer Agents from Natural Products, CRC Press, (2005) ISBN:9780849318634; and David E. Thurston, Chemistry and Pharmacology of Anticancer Drugs, CRC Press, (2006) ISBN 9780849392191.

Exemplary of the bioconjugate function in accordance with embodiments of the present invention are targeted delivery system where the agent or compound to be delivered may be conjugated or otherwise attached to cobalamin without affecting the cobalamin's ability to bind to the appropriate receptor(s). Therefore, it is often the case that the receptor-binding domain(s) of the cobalamin are not modified. Likewise, for successful targeted delivery the agent or compound should be released from the cobalamin in a therapeutically effective form and at the right location. Some event, substance, or condition should be present in the targeted location that will cause the agent to separate from the carrier. Successful methods of drug targeting can involve agent-cobalamin linkages that are sensitive to particular conditions or processes that are prevalent in the target location.

As used herein, the term “covalent linkage” or “covalent bond” refers to an atom or molecule which covalently or coordinate covalently binds together two components. With regard to the present invention, a covalent linkage is intended to include atoms and molecules which can be used to covalently bind a taxane to the cobalt atom of cobalamin. Though not excluded, it is preferred that the covalent linkage not prevent the binding of cobalamin to its transport proteins, either by sterically hindering interaction between cobalamin and the protein, or by altering the binding domain of cobalamin in such a way as to render it conformationally incompatible with the protein. Likewise, preferably, the covalent linkage should not act in these ways to prevent the binding of the cobalamin-transport protein complex with cobalamin receptors.

As used herein, the term “metronomic dosing” generally refers to a long-term, low-dose, frequent administration of oral chemotherapeutic drugs. For example, one metronomic dosing therapy can comprise administering approximately one-fourth of the standard dose of a traditionally twenty-one day chemotherapy regime (one fourth of what you would have received on day one) and dividing that dose over the twenty-one day chemotherapy period. Generally, the amount to be administered is one that may not kill tumor cells, but it is enough to prevent the formation of new blood vessels a process called anti-angiogenesis (the formation of blood vessels is called angiogenesis). As such, the amount to be administered for in any given metronomic dosing therapy can vary. New blood vessels are formed by the migration of circulating endothelial cells to the site of the tumor where further recruitment takes place. Metronomic or low frequent dosing can reduce the toxic side effects of traditional chemotherapy, because the dose that is chosen is far below the range that produces toxic side effects. In addition, since the patient is receiving frequent low dose amounts of the therapeutic drug with out the traditional break in chemotherapy, the endothelial cells which are migrating to the tumor are now targeted by the chemotherapeutic and killed usually as a result of apoptosis. The end result is that there is no formation of functioning blood vessels; thus, the tumor is starved for nutrients and dies

As used herein, the term “maximum tolerated dose” or “MTD” refers to the highest dose of an anti-cancer agent during chemotherapy that when administered to a subject will be effective against a tumor but does not produce excessive toxicity (side-effects, e.g., neutropenia, neurologic disorders, rash, fever etc.) intolerable to the subject. Generally, an MTD is subject specific and is adjusted for the patient's body surface area; a measurement that correlates with blood volume. Ultimately, the MTD can be determined by those having the requisite skill and experience, such as an oncologist.

As used herein, the term “angiogenesis” or “angiogenic” refers to a physiological process involving the growth of new blood vessels. The growth of new blood vessels is an important natural process occurring in the body, both in health and in disease. In regards to tumors, the term “anti-angiogenic” refers to those compounds or agents that inhibit the growth of new blood vessels, effectively cutting off the existing blood supply of the tumor(s). For example, such anti-angiogenic compounds include, but are not limited to, bevacizumab, suramin, sunitinib, thalidomide, tamoxifen, vatalinib, cilenigtide, celecoxib, erlotinib, lenalidomide, ranibizumab, pegaptanib, sorafenib, and mixtures thereof.

As used herein, the term “cancer” refers to a class of diseases or disorders characterized by uncontrolled division of cells and the ability of these cells to spread, either by direct growth or proliferation into adjacent tissue through invasion, or by implantation into distant sites by metastasis (where cancer cells are transported through the bloodstream or lymphatic system). Various types of cancers include, but are not limited to, adrenocortical cancer, basal cell carcinoma (skin), bladder cancer, bowel cancer, brain and CNS tumors, breast cancer, carcinoid tumors, cervical cancer, chondrosarcoma, choriocarcinoma, colorectal cancers, endocrine cancers, endometrial cancer, esophageal cancer, Ewing's sarcoma, eye cancer, gastric cancer, gastrointestinal cancers, genitourinary cancers, glioma, gynaecological cancers, head and neck cancer, hepatocellular cancer, Hodgkin's disease, hypopharynx cancer, islet cell cancer, Kaposi's sarcoma, kidney cancer, laryngeal cancer, leukaemia, liver cancer, lung cancer, lymphoma, melanoma, mesothelioma, myeloma (multiple), nasopharyngeal cancer, neuroblastoma, non Hodgkin's lymphoma, non-melanoma skin cancer, oesophageal cancer, osteosarcoma, ovarian cancer, pancreas cancer, pituitary cancer, prostate cancer, renal cell carcinoma, retinoblastoma, rhabdomyosarcoma, sarcoma, skin cancer, squamous cell carcinoma (skin), stomach cancer, testicular cancer, thymus cancer, thyroid cancer, transitional cell cancer (bladder), trophoblastic cancer, uterus cancer, and vaginal cancer.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 micron to about 5 microns” should be interpreted to include not only the explicitly recited values of about 1 micron to about 5 microns, but also include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

In accordance with these definitions, the present invention provides methods and compositions having anti-cancer compounds in which a taxane or derivative can be covalently bound to the cobalt atom of a cobalamin. It is noted that when discussing a cobalamin-taxane bioconjugate containing composition or a method of administering such a composition, each of these discussions can be considered applicable to other embodiments describe herein, whether or not they are explicitly discussed in the context of that embodiment. Thus, for example, in discussing taxanes from the anti-cancer compositions, those taxanes can also be used in the method for administering anti-cancer compositions, and vice versa.

In one embodiment, an anti-cancer compound can comprises a taxane covalently bonded to a cobalt atom of a cobalamin. In another embodiment, a method of orally delivering a taxane can comprise orally administering to a subject a cobalamin-taxane bioconjugate, where the bioconjugate has a taxane covalently attached to a cobalt atom of a cobalamin. In yet another embodiment, a method of treating a cancer can comprise administering to a subject a therapeutically effective amount of an anti-cancer compound including a taxane covalently bonded to a cobalt atom of a cobalamin. In still yet another embodiment, a method of reducing blood flow to a cancerous tumor in a subject can comprise administering an anti-angiogenic compound to a subject with a tumor, wherein the compound comprises a taxane covalently bonded to a cobalt atom of a cobalamin. Generally, attaching the taxane to the cobalt atom of cobalamin more closely approximates the binding arrangement seen in stable, biologically active forms of cobalamin, such as adenosylcobalamin. It has been recognized that the attachment of a taxane to the cobalt atom of a cobalamin can significantly increase the water solubility of the taxane as a cobalamin-taxane bioconjugate.

As such, the compositions and methods of the present invention provide a cobalamin-taxane bioconjugate that can be water soluble. Generally, taxanes are insoluble in water. For example, paclitaxel has a water solubility of less than 0.004 mg/ml. However, when conjugated to a cobalt atom of a cobalamin, as shown in the following structure and described herein, a cobalamin-paclitaxel bioconjugate can have water solubility of over 100 mg/ml. As such, in one embodiment, a cobalamin-taxane bioconjugate can have a water solubility of at least 0.5 mg/ml. In another embodiment, a cobalamin-taxane bioconjugate can have a water solubility of at least 10 mg/ml. In yet another embodiment, the water solubility can be at least 50 mg/ml. In still yet another embodiment, the water solubility can be at least 100 mg/ml. As such, the cobalamin-taxane bioconjugates provided herein can be orally administered to a subject. Specifically, the cobalamin-taxane bioconjugate can be a cobalamin-paclitaxel bioconjugate having the following structure:

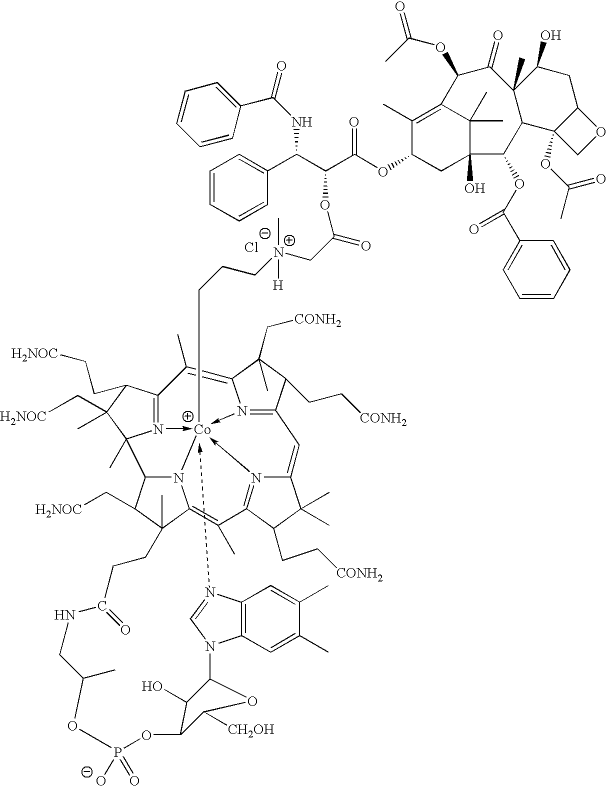

Alternatively, the cobalamin-taxane bioconjugate can be a cobalamin-docetaxel bioconjugate having the following structure:

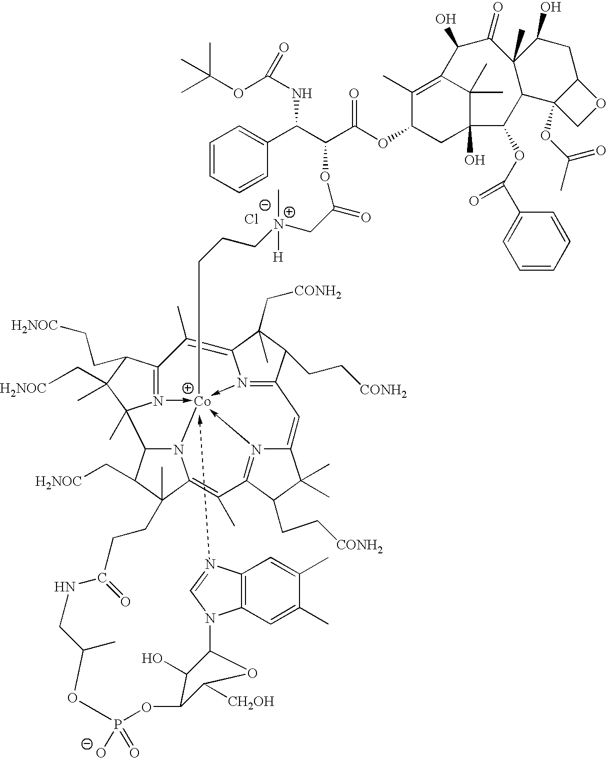

In each of the two above structures as well as in other similar embodiments, it is understood that although the Cl⁻ counter ion is shown, other similar pharmaceutically acceptable counter ions can alternatively be used.

The cobalamin-taxane bioconjugates can have a water solubility several orders of magnitude higher than unconjugated taxanes. In one embodiment, the cobalamin-taxane bioconjugate can have at least a 10 fold increase in water solubility compared to the unconjugated taxane. In another embodiment, the increase can be at least 100 fold. In yet another embodiment, the increase can be at least 1000 fold.

Additionally, it has been recognized that the cobalamin-taxane bioconjugates disclosed herein can have increased bioavailability in a subject. Bioavailability of a compound can be dependent on P-Glycoprotein (P-gp), an ATP-dependent drug pump, which can transport a broad range of hydrophobic compounds out of a cell. This can lead to the phenomenon of multi-drug resistance. Expression of P-gp can be quite variable in humans. Generally, the highest levels can be found in the apical membranes of the blood-brain/testes barrier, intestines, liver, and kidney. Over-expression in cancer patients can undermine chemotherapy as the drug is pumped out via this pump. P-gp can also affect the penetration of the drug to solid tumors. Additionally, in HIV patients, it has been shown that P-gp in the intestine affects the therapeutic levels of drugs in these patients. P-gp has been shown to affect the ability of taxanes, such as paclitaxel or docetaxel, to enter the cells and become bioavailable. Therefore, the bioconjugates of the present invention can be structurally different as to bypass the P-gp pathway leading to increased bioavailability of the bioconjugate. Additionally, cobalamin bioconjugates can use a facultative transport mechanism, which would also bypass the P-gp pathway leading to increased bioavailability.

The taxane for use can be selected from the group consisting of paclitaxel and docetaxel, derivatives thereof, and mixtures thereof. In one embodiment, the taxane can be paclitaxel. The cobalamin can be selected from the group consisting of cyanocobalamin including anilide, ethylamide, proprionamide, monocarboxylic, dicarboxylic, and tricarboxylic acid derivatives thereof; hydroxycobalamin including anilide, ethylamide, proprionamide, monocarboxylic, dicarboxylic, and tricarboxylic acid derivatives thereof; methylcobalamin including anilide, ethylamide, proprionamide, monocarboxylic, dicarboxylic, and tricarboxylic acid derivatives thereof; adenosylcobalamin including anilide, ethylamide, proprionamide, monocarboxylic, dicarboxylic, and tricarboxylic acid derivatives thereof; aquocobalamin; cyanocobalamin carbanalide; desdimethyl cobalamin; monoethylamide cobalamin; methlyamide cobalamin; 5′-deoxyadenosylcobalamin; cobamamide derivatives; chlorocobalamin; sulfitocobalamin; nitrocobalamin; thiocyanatocobalamin; benzimidazole derivatives including 5,6-dichlorobenzimidazole, 5-hydroxybenzimidazole, trimethylbenzimidazole, as well as adenosylcyanocobalamin; cobalamin lactone; cobalamin lactam; 5-O— methylbenzylcobalamin; derivatives thereof; mixtures thereof; and analogues thereof wherein the cobalt is replaced by another metal. In one embodiment, the cobalamin can be one of the vitamin B₁₂ types of cobalamin, and in one specific embodiment, hydroxycobalamin.

Specifically, the present invention relates to solubilization and oral drug delivery of taxanes and their derivatives to various cancer cells and/or tumors via a cobalamin-taxane bioconjugate. In addition, it is noted that there may be an inherent targeting effect via the cobalamin molecule. When introduced into the bloodstream or gastrointestinal tract of a subject, such a bioconjugate can take advantage of existing systems for absorption, transport, and binding of cobalamin. In this way, the taxane can be transported to cells that bear receptors for cobalamin and be taken up by those cells. As noted above, some cells or cell populations in a given subject can utilize cobalamin more heavily at a given time than other cells; consequently expression of cobalamin receptors is upregulated in such cells at those times. Thus, when the bioconjugate is administered to a subject, more of the taxane can be taken up by these cells than by other cells. Thus, the present invention provides a method for concentrating a taxane to sites where cells are utilizing cobalamin heavily. Increased demand for cobalamin is associated with, among other things, rapid cellular proliferation. Therefore, the present invention can be used to concentrate taxanes in neoplastic cells in a subject suffering from a proliferative disease, such as cancer.

Taxanes have been used to produce various chemotherapy drugs. The principal mechanism of the taxane class of drugs is the inhibition of the microtubule function. Taxanes can stabilize guanosine diphosphate (GDP)-bound tubulin in the microtubule. This stabilization results in what is commonly referred to as a “frozen mitosis.” As microtubules are essential to cell division, such inhibition provides an effective treatment of various cancers. Additional information regarding the mechanisms for taxane can be found in “In the G2/M Phase” Allman et al., British J. Cancer Research (2003) 88, 1649-1658, which is incorporated by reference. Such cancers include, but are not limited to, adrenocortical cancer, basal cell carcinoma (skin), bladder cancer, bowel cancer, brain and CNS tumors, breast cancer, carcinoid tumors, cervical cancer, chondrosarcoma, choriocarcinoma, colorectal cancers, endocrine cancers, endometrial cancer, esophageal cancer, Ewing's sarcoma, eye cancer, gastric cancer, gastrointestinal cancers, genitourinary cancers, glioma, gynaecological cancers, head and neck cancer, hepatocellular cancer, Hodgkin's disease, hypopharynx cancer, islet cell cancer, Kaposi's sarcoma, kidney cancer, laryngeal cancer, leukaemia, liver cancer, lung cancer, lymphoma, melanoma, mesothelioma, myeloma (multiple), nasopharyngeal cancer, neuroblastoma, non Hodgkin's lymphoma, non-melanoma skin cancer, oesophageal cancer, osteosarcoma, ovarian cancer, pancreas cancer, pituitary cancer, prostate cancer, renal cell carcinoma, retinoblastoma, rhabdomyosarcoma, sarcoma, skin cancer, squamous cell carcinoma (skin), stomach cancer, testicular cancer, thymus cancer, thyroid cancer, transitional cell cancer (bladder), trophoblastic cancer, uterus cancer, and vaginal cancer. In one embodiment, the cancer can be renal/kidney cancer. In another embodiment, the cancer can be colon cancer. In yet another embodiment, the cancer can be prostate cancer. In still yet another embodiment, the cancer can be breast cancer.

The taxane can be covalently bonded to the cobalt atom directly or through a covalent linkage. The linkage serves as a connection between the cobalamin and the taxane, and can serve to achieve a desired distance between these two components, while preferably not negatively affecting the binding of the bioconjugate to proteins involved in cobalamin metabolism. In particular, the linkage can include an ester linkage. Alternatively or additionally, the linkage can include a quaternary amine. In another alternative embodiment, the linkage could be a hydrazone linkage. The bioconjugate of the present invention can also include a linkage comprising a polymethylene, carbonate, ether, acetal, or any combination of these units. In a more general embodiment that that shown above, the cobalamin-taxane bioconjugate can be linked as follows:

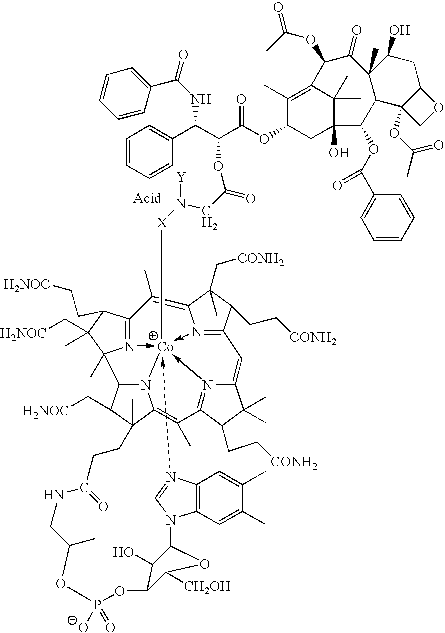

where Y is any alkyl containing 1 to 4 carbons; and X is an optionally substituted, saturated, branched, or linear, C₁₋₅₀ alkylene, cycloalkylene or aromatic group, optionally with one or more carbons within the chain being replaced with, N, O or S, and wherein the optional substituents are selected from carbonyl, carboxy, hydroxyl, amino and other groups. The “Acid” can be any organic or inorganic acid, preferably having the ability to form pharmaceutically acceptable salts. Other linkages that will serve the functions described above will be known to those having skill in the art, and are encompassed by the present invention.

Such a linkage can serve as a target for an enzyme that will cleave the linkage, releasing the taxane from the cobalamin. Such an enzyme can be present in the subject's bloodstream and thereby release the taxane into the general circulation, or it can be localized specifically to a site or cell type that is the intended target for delivery of the taxane. Alternatively, the linkage can be of a type that will cleave or degrade when exposed to a certain environment or, particularly, a characteristic of that environment such as a certain pH range or range of temperatures. The linkage can be of a “self-destructing” type, i.e. it will be consumed in the process of cleavage, so that said cleavage will yield only the original cobalamin and the taxane molecules absent any remaining large sections of the linkage. Those having skill in the art will recognize other release mechanisms derived from various linkages that can be used in accordance with the present invention.

The compounds of the present invention can be further administered as pharmaceutical compositions in treating various cancers. Such a composition can further comprise one or more excipients, including binders, fillers, lubricants, disintegrants, flavoring agents, coloring agents, sweeteners, thickeners, coatings, and combinations thereof. The composition of the present invention can be formulated into a number of dosage forms including syrups, elixirs, solutions, suspensions, emulsions, capsules, tablets, lozenges, and suppositories. Differing administration regimens will call for different dosage forms, depending on factors such as the subject's age, medical condition, level of need for treatment, as well as the desired time course of therapeutic effect. Those having skill in the art will recognize that various classes of excipients can each provide different characteristics to a pharmaceutical composition and that they can be combined in certain ways in accordance with the present invention to achieve an appropriate dosage form. The present invention provides compounds that can be administered to a subject orally, dermally, or parenterally.

One aspect of the present invention is that administering the bioconjugate can be more effective in treating cancer than administering the taxane and the cobalamin as separate molecules. In light of the fact that taxanes alone can provide anti-angiogenic effects, the present invention provides cobalamin-taxane bioconjugates as anti-angiogenic compounds for treating various cancers. The amount of taxane to cobalamin can generally be equal, e.g., the taxane to cobalamin molar ratio can about 1:1. However, the anti-cancer composition can have an excess of cobalamin or taxane that is not covalently bonded. In one embodiment, a composition can have a cobalamin to cobalamin-taxane bioconjugate molar ratio from about 1.2:1 to about 10:1. Additionally, the bioconjugate can further include additional anti-angiogenic compounds. Such additional anti-angiogenic compounds include, but are not limited to, bevacizumab, suramin, sunitinib, thalidomide, tamoxifen, vatalinib, cilenigtide, celecoxib, erlotinib, lenalidomide, ranibizumab, pegaptanib, sorafenib, and mixtures thereof.

The compositions of the present invention can also include additional anti-cancer compounds not covalently attached to the cobalamin. Such additional anti-cancer compounds include, but are not limited to, cyclophosphamide, 5-fluorouracil, fluoruracil, doxorubicin, iridotecan, methotrexate, mercaptopurine, daunorubicin, etoposide, vinblastine, gemcitabine, vincristine, erlotinib, capecitabine, carboplatin, ifosfamide, imatinib mesylate, irinotecan, letrozole, leucovorin, mitomycin C, mitoxantrone, pamidronate, panitumumab, tamoxifen, thalidomide, topotecan, trastuzumab, and mixtures thereof. Additionally, other cancer compounds and anti-angiogenic compounds are contemplated by the methods and compositions of the present invention including, but not limited to, those found in Gordon M. Cragg, David G. I. Kingston, & David J. Newman, Anticancer Agents from Natural Products, CRC Press, (2005) ISBN:9780849318634; and David E. Thurston, Chemistry and Pharmacology of Anticancer Drugs, CRC Press, (2006) ISBN 9780849392191, both of which are incorporated by reference in their entireties.

Therefore, the present invention provides compositions having anti-cancer compounds and cobalamin-taxane bioconjugates. Such compositions can have an anti-cancer compound to a cobalamin-taxane bioconjugate molar ratio from about 10:1 to about 1:10. In one embodiment, the ratio can be about 5:1 to about 1:5.

As previously discussed, cancer treatment is one area that can benefit from using cobalamin as a drug delivery vehicle. Also, as rapidly dividing cells require cobalamin for thymidine synthesis in DNA replication, cobalamin receptors are highly upregulated in rapidly proliferating tumor cells. This makes cobalamin a useful vehicle to preferentially deliver drugs to cancer cells. The possible benefits are most apparent in conventional chemotherapy, where effective targeting can strengthen the attack on tumor cells while lessening the damage to benign cells. As such, the cobalamin-taxane bioconjugates can be administered in maximum tolerated doses as used in conventional chemotherapy. However, as anti-angiogenic chemotherapy has been studied and developed, the cobalamin-taxane bioconjugates can be used effectively in these chemotherapy regimes as well, especially since the present invention has provided methods and compositions that enable oral delivery of taxanes through bioconjugation with cobalamin which is a significant advancement in the art. As such, the cobalamin-taxane bioconjugates can be administered by metronomic dosing. In one embodiment, administering the bioconjugates of the present invention can be used to achieve serum levels in a subject of about 0.1 ng/ml to about 20,000 ng/ml. Further, the taxanes of the cobalamin-taxane bioconjugates of the present invention can be administered at about 1 mg/kg/day to about 10 mg/kg/day. In one embodiment, the rate can be about 2 mg/kg/day to about 6 mg/kg/day.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

### EXAMPLES

The following provides examples of oral taxanes in accordance with the compositions and methods previously disclosed. Additionally, some of the examples include studies performed showing the effects of oral taxanes on animals in accordance with embodiments of the present invention.

### Example 1

### Preparation of Cobalamin-Paclitaxel Bioconjugate

A cobalamin-paclitaxel bioconjugate was prepared using the following reaction schematic:

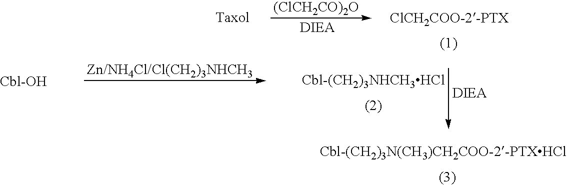

### ABBREVIATIONS

Cbl-: α-substituted cobalamin
PTX: paclitaxel
DIEA: diisopropylethylamine

Specifically, a Waters Alliance 2695 HPLC system and a 2996 PDA detector are used for this analytical work. 50 mM H₃PO₄ (adjusted to pH 3.0 with ammonia) (buffer A) and acetonitrile/water (9:1) (buffer B) are used as aqueous and organic eluents, respectively, unless stated otherwise. Waters Delta-Pak C₁₈ 15 μm 100 Å 3.9×300 mm column (P/N WAT011797) and 1 mL/min flow rate are also used. Mass spectra is acquired on PE-Sciex API 2000 Mass Spectrometer.

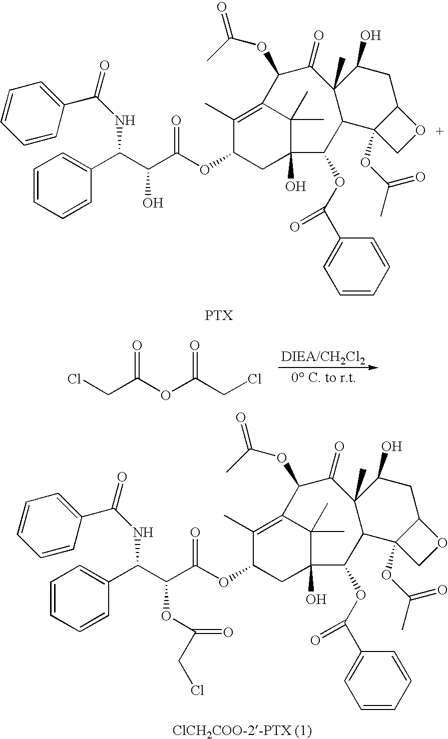

To a stirred solution containing paclitaxel (1.074 g, 1.258 mmol) in CH₂Cl₂ (7 ml) is added 2-chloroacetic anhydride (0.236 g, 1.376 mmol) and DIEA (0.26 ml, 1.376 mmol) consequently at 0° C. The reaction is slowly warmed up to room temperature. After 24 hrs, the reaction mixture is concentrated purified by flash chromatography (silica gel, 0-80% ethyl acetate in hexane) and 0.987 g (84.33%) of white solid is obtained.

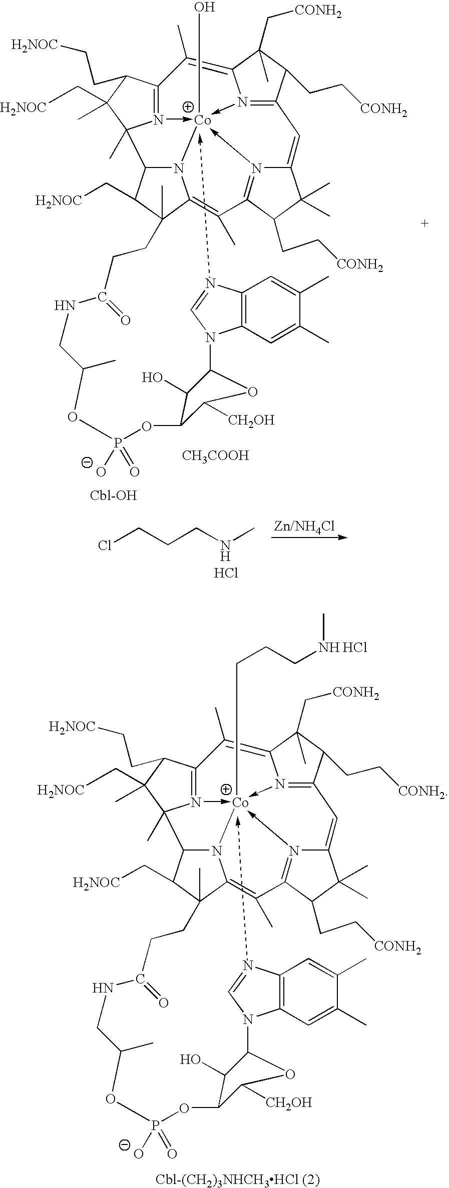

Hydroxocobalamin acetate (0.5 g, 0.355 mmol) is dissolved in DI H₂O (25 ml), and N-methyl-3-chloropropylamine (0.108 g, 0.751 mmol) and NH₄Cl (0.195 mg, 3.63 mmol) is added to the solution. The solution is degassed by bubbling with N₂ for 30 min. Then, Zn dust (<10 micron) (0.238 g, 3.63 mmol) is added in one portion. All the starting material is consumed after the reaction is stirred under N₂ for 3.5 h. The reaction mixture is then filtered with Whatman No. 42 filter paper to remove Zn. The filtrate is loaded on a Waters C18 Sep-Pak cartridge (10 g of C₁₈ sorbant) that is pre-washed by washing with 60 ml of methanol followed with 100 ml of water. All salts are removed from the cartridge with 100 ml of water and the product is eluted with CH₃OH—H₂O (9:1) and concentrated to dry. The residue is resuspended in 4 ml of methanol and precipitated in 100 mL of 1:1 (V/V) CH₂Cl₂/anhydrous Et₂O. The red solid is filtered and washed with acetone (20 ml) and ether (20 ml), affording 0.482 g (yield 94.6%, purity 98%) of product.

Cbl—(CH₂)₃N(CH₃)CH₂COO-2′-PTX (3)

A solution of compound 1 (0.743 g, 0.799 mmol, 1.0 eq), 2 (1.976 g, 1.374 mmol, 1.72 eq), and DIEA (0.24 ml, 1.374 mmol, 1.72 eq) in DMSO (48 mL) is stirred at room temperature for 3 days. HPLC indicated starting material 1 is consumed. The reaction mixture is added to stirring CH₂Cl₂/ether (1:2, 450 ml). The resulting precipitate is collected, washed with CH₂Cl₂ (20 ml×3) and ether (20 ml×3), and air-dried. The crude product is diluted with 0.01; N HCl (200 ml) and applied to a C₁₈ reverse phase 43 g column which is pre-washed sequentially with 7 volumes of methanol and water. The column is first washed with water (50 ml) and eluted with 5-40% B in buffer A (200 ml each with 5% increment). The fractions are checked for purity by HPLC. The desired fractions are combined, diluted with one volume of water, and adsorbed onto a Waters C18 Sep-Pak cartridge (10 g, P/N WAT043350, pre-washed sequentially with 3 volumes of methanol and water). The product is washed with water (20 mL×3), 0.01 M HCl (20 mL×3), water (20 mL×3) and eluted off the cartridge with 9:1 acetonitrile/water (50 mL). The organic solvent is removed with a rotary evaporator. The residue is dissolved in 0.01 N hydrochloride solution (40 mL, with the aid of a few drops of 0.1 N hydrochloride solution), filtered by 0.45 μm NYLON membrane filter, and lyophilized. 780 mg (41.9%) of red powder is obtained. ES(+)-MS: 1148.9 [(M+H)²⁺], 1329.9 (Cbl⁺), 665.7 [(Cbl+H)²⁺], 971.6 [(Cbl-359)+], 359.1 (fragment from the breakdown of C—OP(O) bond). HPLC indicates that the product is about 98.6% pure.

The resultant compound has the following structure:

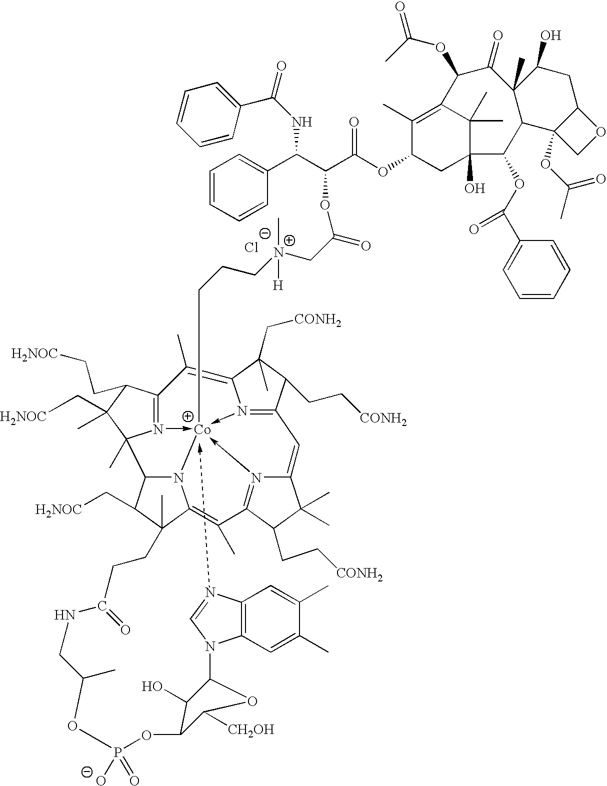

### Example 2

### Preparation of Cobalamin-Docetaxel Bioconjugate

Similar procedures are followed as outlined in Example 1, but with docetaxel as the principal taxane, resulting in the following structure:

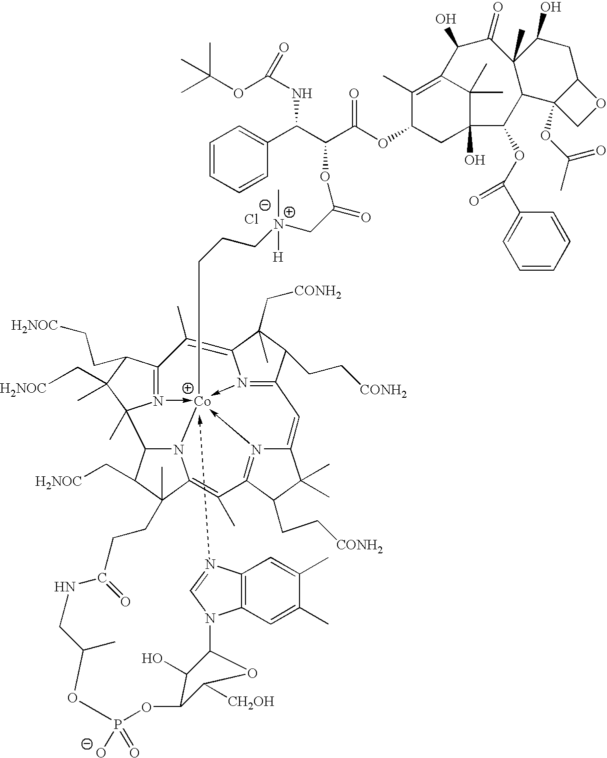

### Example 3-Cobalamin-Paclitaxel Bioconjugate Dose Study

A group of 6 mice are administered various dosages of the cobalamin-paclitaxel bioconjugate prepared in accordance with Example 1 over a 28 day period. The effects to the viable circulating endothelial cell precursors and white blood cells are measured after 28 days. Corresponding amounts of the cobalamin-paclitaxel bioconjugate, viable circulating endothelial cell precursors, and white blood cells are presented in the Table 1:

TABLE 1

| Amount of paclitaxel | | |
|---|---|---|
| delivered as a cobalamin- | Viable CEPs per | White blood cells |
| paclitaxel bioconjugate | microliter of | per 10⁴ peripheral |
| (paclitaxel in mg/kg) | peripheral blood | blood cells |
| 0.0 (control) | 1.5 | 6800 |
| 30 | 1.2 | 8100 |
| 6 | 0.9 | 6700 |
| 3 | 0.4 | 7000 |
| 2 | 0.25 | 6700 |
| 1.5 | 0.4 | 6700 |

As can be seen from Table 1, administration of the cobalamin-paclitaxel bioconjugate has an anti-angiogenic effect (marked decrease in viable CEPs) at each dose. However, the most effective dose is not proportional to the amount of paclitaxel administered. In fact, the most effective dose in this particular study is about 2 mg/kg. Furthermore, the absence of a decrease in the white blood cell count shows that such a dosage is less toxic to the mouse (no neutropenia).

### Example 4

### Anti-Angiogenic Efficacy of Cobalamin-Paclitaxel Bioconjugate by Matrigel Plug Perfusion Assay

A Matrigel plug perfusion in vivo assay is performed to determine the anti-angiogenic efficiacy of the cobalamin-paclitaxel bioconjugate (Cob-Pac) of Example 1. The assay uses Matrigel, a gelatinous protein mixture secreted by mouse tumor cells and marketed by BD Biosciences, to duplicate tissue environments. Matrigel is liquid at room temperature, but when injected into the animal, forms a solid plug. If a growth vessel stimulant such as basic fibroblast growth factor (bFGF) is mixed with the Matrigel, the bFGF stimulates the formation of new blood vessel in the plug, which can be monitored in the animal via fluorescence techniques. In the current study, Matrigel is injected either alone or with bFGF subcutaneously into mice. Then, as indicated in Table 2, groups of mice are either treated by oral gavage with the cobalamin-paclitaxel conjugate or in the last group with the mouse anti-VEGF receptor antibody, DC101. DC101 is viewed by many as the gold standard for anti-angiogenesis in the mouse. The results are shown in Table 2:

TABLE 2

| | Matrigel Plug/Plasma |
|---|---|
| Assay | Fluorescence Ratio |
| Water with Matrigel | 0.00050 |
| Water with Matrigel and bFGF | 0.00125 |
| Cob-Pac with Matrigel and bFGF | 0.00110 |
| (30 mg/kg expressed in paclitaxel units) | |
| Cob-Pac with Matrigel and bFGF | 0.00050 |
| (6 mg/kg expressed in paclitaxel units) | |
| Cob-Pac with Matrigel and bFGF | 0.00070 |
| (2 mg/kg expressed in paclitaxel units) | |
| DC101 with Matrigel and bFGF | 0.00072 |
| (800 μg/kg) | |

As can be seen, the addition of bFGF stimulated the growth of blood vessels on the Matrigel assay as indicated by the fluorescence ratio in the matrigel plus bFGF. The addition of cobalamin-paclitaxel bioconjugate inhibited the growth of new blood vessels in each instance. However, the greatest effect was at the 2 mg/kg (expressed in paclitaxel units) and 6 mg/kg (expressed in paclitaxel units) doses. The cobalamin-paclitaxel bioconjugate provided better performance than that of DC101, an effective rodent specific anti-angiogenic compound that is well known in the art.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A bioconjugate, comprising a taxane covalently bonded to a cobalt atom of a cobalamin.

2. The bioconjugate of claim 1, wherein the taxane includes a member selected from the group consisting of paclitaxel and docetaxel, derivatives thereof, and mixtures thereof.

3. The bioconjugate of claim 2, wherein the taxane is paclitaxel.

4. The bioconjugate of claim 2, wherein the taxane is docetaxel.

5. The bioconjugate of claim 1, wherein the cobalamin includes a member selected from the group consisting of cyanocobalamin including anilide, ethylamide, proprionamide, monocarboxylic, dicarboxylic, or tricarboxylic acid derivatives thereof; hydroxycobalamin including anilide, ethylamide, proprionamide, monocarboxylic, dicarboxylic, or tricarboxylic acid derivatives thereof; methylcobalamin including anilide, ethylamide, proprionamide, monocarboxylic, dicarboxylic, or tricarboxylic acid derivatives thereof; adenosylcobalamin including anilide, ethylamide, proprionamide, monocarboxylic, dicarboxylic, or tricarboxylic acid derivatives thereof; aquocobalamin; cyanocobalamin carbanalide; desdimethyl cobalamin; monoethylamide cobalamin; methlyamide cobalamin; 5′-deoxyadenosylcobalamin; cobamamide derivatives; chlorocobalamin; sulfitocobalamin; nitrocobalamin; thiocyanatocobalamin; benzimidazole derivatives including 5,6-dichlorobenzimidazole, 5-hydroxybenzimidazole, trimethylbenzimidazole, or adenosylcyanocobalamin; cobalamin lactone; cobalamin lactam; 5-o-methylbenzylcobalamin; derivatives thereof; mixtures thereof; and analogues thereof.

6. The bioconjugate of claim 1, wherein the cobalamin is a hydroxycobalamin.

7. The bioconjugate of claim 1, wherein the cobalamin is vitamin B₁₂.

8. The bioconjugate of claim 1, formulated in a composition for oral delivery.

9. The bioconjugate of claim 1, formulated in a composition for parenteral delivery.

10. The bioconjugate of claim 1, formulated in a composition for dermal delivery.

11. The bioconjugate of claim 1, wherein the bioconjugate has water solubility of at least 50 mg/ml.

12. The bioconjugate of claim 1, wherein the bioconjugate has a water solubility of at least 100 mg/ml.

13. The bioconjugate of claim 1, formulated in a composition having a taxane to cobalamin molar ratio of about 1:1.

14. The bioconjugate of claim 1, formulated in a composition with an anti-cancer compound not covalently attached to the cobalamin.

15. The bioconjugate of claim 14, wherein the anti-cancer compound includes a member selected from the group consisting of cyclophosphamide, 5-fluorouracil, fluoruracil, doxorubicin, iridotecan, methotrexate, mercaptopurine, daunorubicin, etoposide, vinbiastine, gemcitabine, vincristine, erlotinib, capecitabine, carboplatin, ifosfamide, imatinib mesylate, irinotecan, letrozole, leucovorin, mitomycin C, mitoxantrone, pamidronate, panitumumab, tamoxifen, thalidomide, topotecan, trastuzumab, and mixtures thereof.

16. The bioconjugate of claim 14, wherein the composition has an anti-cancer compound to bioconjugate molar ratio from about 10:1 to about 1:10.

17. The bioconjugate of claim 1, wherein the bioconjugate is present in a composition with an excess of cobalamin that is not covalently bonded to the taxane.

18. The bioconjugate of claim 17, wherein the composition has a cobalamin to bioconjugate molar ratio from about 1.2:1 to about 10:1.

19. The bioconjugate of claim 1, formulated in a composition with an anti-angiogenic compound not covalently attached to the cobalamin.

20. The bioconjugate of claim 19, wherein the anti-angiogenic compound is selected from the group consisting of bevacizumab, suramin, suhitinib, thalidomide, tamoxifen, vatalinib, cilenigtide, celecoxib, erlotinib, lenalidomide, ranibizumab, pegaptanib, sorafenib, and mixtures thereof.

21. The bioconjugate of claim 1, wherein the taxane is covalently bonded to the cobalamin through an ester linkage.

22. The bioconjugate of claim 1, wherein the taxane is covalently bonded to the cobalamin through a quaternary amine.

23. The bioconjugate of claim 1, wherein the taxane covalently bonded to the cobalt atom of the cobalamin is paclitaxel covalently bonded to the cobalt atom of a hydroxycobalamin.

24. A cobalamin-paclitaxel bioconjugate, comprising the structure:

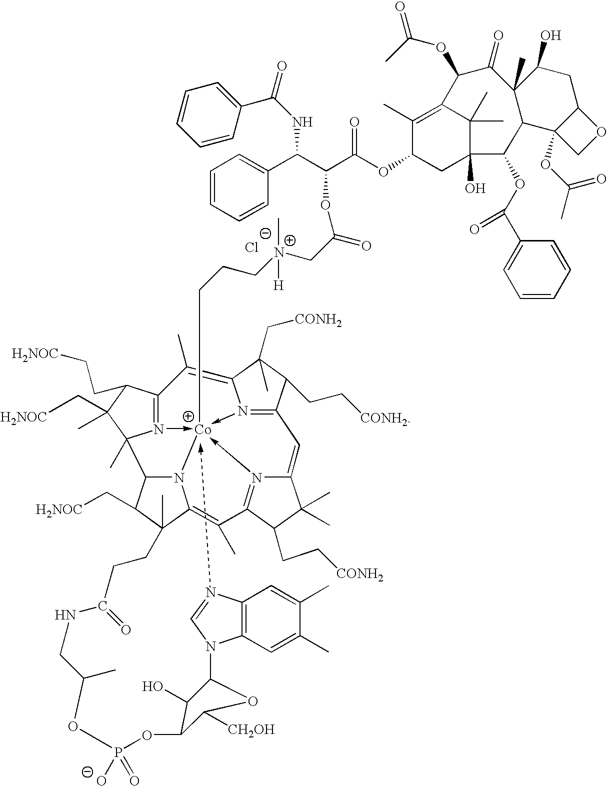

25. The bioconjugate of claim 24, wherein the water solubility of the cobalamin-paclitaxel bioconjugate is at least 50 mg/ml.

26. The bioconjugate of claim 24, wherein the water solubility of the cobalamin-paclitaxel bioconjugate is at least 100 mg/ml.

27. The bioconjugate of claim 24, wherein the bioconjugate is formulated in a composition for oral administration to a subject.

28. The bioconjugate of claim 24, wherein the bioconjugate is formulated in a composition for treatment of cancer.

29. A cobalamin-docetaxel bioconjugate, comprising the structure:

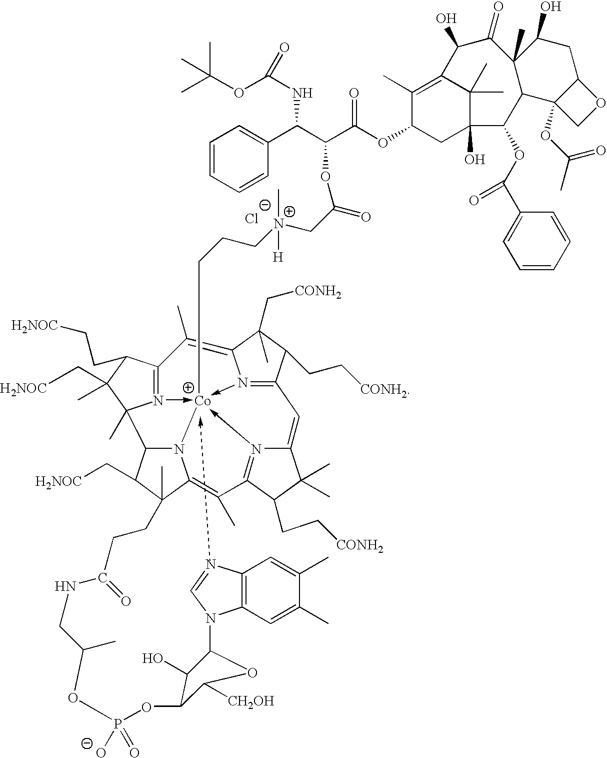

30. The bioconjugate of claim 29, wherein the water solubility of the cobalamin-docetaxel bioconjugate is at least 50 mg/ml.

31. The bioconjugate of claim 29, wherein the water solubility of the cobalamin-docetaxel bioconjugate is at least 100 mg/ml.

32. The bioconjugate of claim 29, wherein the bioconjugate is formulated in a composition for oral administration to a subject.

33. The bioconjugate of claim 29, wherein the bioconjugate is formulated in a composition for treatment of cancer.

34. A method of orally delivering a taxane, comprising orally administering to a subject a cobalamin-taxane bioconjugate, wherein the cobalamin-taxane bioconjugate has a taxane covalently bonded to a cobalt atom of a cobalamin, and wherein the water solubility of the cobalamin-taxane bioconjugate is at least 50 mg/ml.

35. The method of claim 34, wherein the water solubility of the cobalamin-taxane bioconjugate is at least 100 mg/ml.

36. The method of claim 34, wherein the cobalamin-taxane bioconjugate comprises the structure:

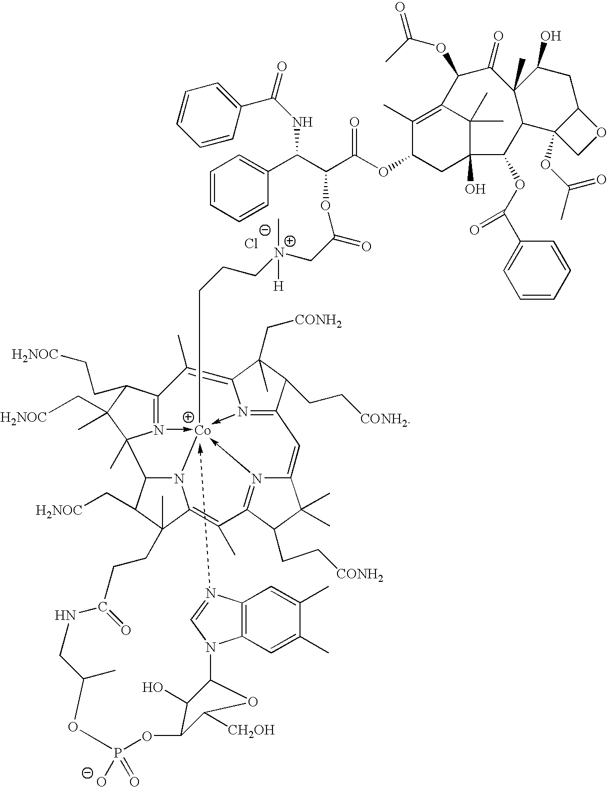

37. The method of claim 34, wherein the cobalamin-taxane bioconjugate comprises the structure:

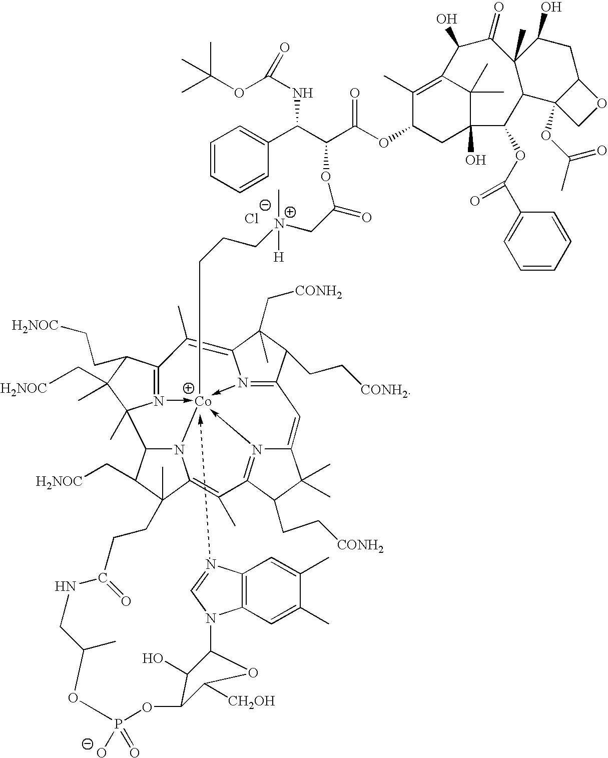

38. A method of treating a cancer, comprising administering to a subject a therapeutically effective amount of a bioconjugate including a taxane covalently bonded to a cobalt atom of a cobalamin.

39. The method of claim 38, wherein the taxane includes a member selected from the group consisting of paclitaxel and docetaxel, derivatives thereof, and mixtures thereof.

40. The method of claim 38, wherein the taxane is paclitaxel.

41. The method of claim 38, wherein the cobalamin includes a member selected from the group consisting of cyanocobalamin including anilide, ethylamide, proprionamide, monocarboxylic, dicarboxylic, and tricarboxylic acid derivatives thereof; hydroxycobalamin including anilide, ethylamide, proprionamide, monocarboxylic, dicarboxylic, and tricarboxylic acid derivatives thereof; methylcobalamin including anilide, ethylamide, proprionamide, monocarboxylic, dicarboxylic, and tricarboxylic acid derivatives thereof; adenosylcobalamin including anilide, ethylamide, proprionamide, monocarboxylic, dicarboxylic, and tricarboxylic acid derivatives thereof; aquocobalamin; cyanocobalamin carbanalide; desdimethyl cobalamin; monoethylamide cobalamin; methlyamide cobalamin; 5′-deoxyadenosylcobalamin; cobamamide derivatives; chlorocobalamin; sulfitocobalamin; nitrocobalamin; thiocyanatocobalamin; benzimidazole derivatives including 5,6-dichlorobenzimidazole, 5-hydroxybenzimidazole, trimethylbenzimidazole, as well as adenosylcyanocobalamin; cobalamin lactone; cobalamin lactam; 5-o-methylbenzylcobalamin; derivatives thereof; mixtures thereof; and analogues thereof.

42. The method of claim 38, wherein the cobalamin is a hydroxycobalamin.

43. The method of claim 38, wherein the cobalamin is vitamin B₁₂.

44. The method of claim 38, wherein the cancer is selected from the group consisting of adrenocortical cancer, basal cell carcinoma, bladder cancer, bowel cancer, brain tumors, CNS tumors, breast cancer, carcinoid tumors, cervical cancer, chondrosarcoma, choriocarcinoma, colorectal cancers, endocrine cancers, endometrial cancer, esophageal cancer, Ewing's sarcoma, eye cancer, gastric cancer, gastrointestinal cancers, genitourinary cancers, glioma, gynaecological cancers, head and neck cancer, hepatocellular cancer, Hodgkin's disease, hypopharynx cancer, islet cell cancer, Kaposi's sarcoma, renal/kidney cancer, laryngeal cancer, leukaemia, liver cancer, lung cancer, lymphoma, melanoma, mesothelioma, myeloma, nasopharyngeal cancer, neuroblastoma, non Hodgkin's lymphoma, non-melanoma skin cancer, oesophageal cancer, osteosarcoma, ovarian cancer, pancreas cancer, pituitary cancer, prostate cancer, renal cell carcinoma, retinoblastoma, rhabdomyosarcoma, sarcoma, skin cancer, squamous cell carcinoma, stomach cancer, testicular cancer, thymus cancer, thyroid cancer, transitional cell cancer, trophoblastic cancer, uterus cancer, vaginal cancer, and combinations thereof.

45. The method of claim 44, wherein the cancer is renal/kidney cancer.

46. The method of claim 44, wherein the cancer is colorectal cancer.

47. The method of claim 44, wherein the cancer is prostate cancer.

48. The method of claim 44, wherein the cancer is breast cancer.

49. The method of claim 38, wherein the step of administering is by oral delivery.

50. The method of claim 38, wherein the step of administering is by parenteral delivery.

51. The method of claim 38, wherein the step of administering is by dermal delivery.

52. The method of claim 38, wherein the step of administering is by metronomic dosing.

53. The method of claim 38, wherein the step of administering is by maximum tolerated dosing.

54. The method of claim 38, wherein the step of administering achieves serum levels of about 0.1 ng/ml to about 20,000 ng/ml of the taxane in the subject.

55. The method of claim 38, wherein the taxane portion of the bioconjugate is administered at about 1 mg/kg/day to about 10 mg/kg/day.

56. The method of claim 38, wherein the taxane portion of the bioconjugate is administered at about 2 mg/kg/day to about 6 mg/kg/day.

57. The method of claim 38, wherein the bioconjugate has a water solubility of at least 50 mg/ml.

58. The method of claim 38, wherein the bioconjugate has a water solubility of at least 100 mg/ml.

59. The method of claim 38, wherein the bioconjugate is an anti-angiogenic compound.

60. The method of claim 38, further comprising coadministering an anti-cancer compound.

61. The method of claim 60, wherein the anti-cancer compound is present in a common composition with the bioconjugate.

62. The method of claim 60, wherein the composition has an anti-cancer compound to bioconjugate molar ratio from about 10:1 to about 1:10.

63. The method of claim 60, wherein the anti-cancer compound includes a member selected from the group consisting of cyclophosphamide, 5-fluorouracil, fluoruracil, doxorubicin, iridotecan, methotrexate, mercaptopurine, daunorubicin, etoposide, vinblastine, gemcitabine, vincristine, erlotinib, capecitabine, carboplatin, ifosfamide, imatinib mesylate, irinotecan, letrozole, leucovorin, mitomycin C, mitoxantrone, pamidronate, panitumumab, tamoxifen, thalidomide, topotecan, trastuzumab, and mixtures thereof.

64. The method of claim 38, wherein the taxane covalently bonded to the cobalt atom of the cobalamin is paclitaxel covalently bonded to the cobalt atom of a hydroxycobalamin.

65. A method of reducing blood flow to a cancerous tumor in a subject, comprising administering an anti-angiogenic compound to a subject with a tumor, said compound comprising a taxane covalently bonded to a cobalt atom of a cobalamin.

66. The method of claim 65, wherein the step of administering is oral.

67. The method of claim 65, wherein the step of administering is parenteral.

68. The method of claim 67, wherein the parenteral delivery is directly into a tumor site.

69. The method of claim 65, wherein the step of administering is dermal.

70. The method of claim 65, wherein the taxane is covalently bonded to the cobalt atom through an ester linkage group.

71. The method of claim 65, wherein the taxane is covalently bonded to the cobalt atom through a linkage group that includes a quaternary amine.

72. The method of claim 65, wherein the step of administering is by metronomic dosing.

73. The method of claim 65, wherein the step of administering is by maximum tolerated dosing.

74. The method of claim 65, wherein the anti-angiogenic compound has a water solubility of at least 50 mg/ml.

75. The method of claim 65, wherein the anti-angiogenic compound has a water solubility of at least 100 mg/ml.

76. The method of claim 65, wherein the taxane covalently bonded to the cobalt atom of the cobalamin is paclitaxel covalently bonded to the cobalt atom of a hydroxycobalamin.
